(19) United States
(12) Patent Application Publication
(10) Pub. No.: US 2009/0131808 A1
(43) Pub. Date: May 21, 2009

(54) SPHYGMOMANOMETER AND CHIP CARD THEREFOR

(75) Inventors: Martin Giersiepen, Muenchen (DE); Dieter Wunder, Schotten (DE); Ulrich Heck, Krefeld (DE); Dirk Freund, Kelkheim (DE); Brigitte Harttmann, Niedernhausen (DE); Stefan Hollinger, Kronberg (DE); Gerrit Roenneberg, Darmstadt (DE); Fred Schnak, Kronberg (DE); Frank Kressmann, Eschborn (DE)

Correspondence Address: DARBY & DARBY P.C., P.O. BOX 770, Church Street Station, New York, NY, 10008-0770, US

(73) Assignee: Kaz USA, Inc., Southborough, MA (US)

(21) Appl. No.: 12/266,750

(22) Filed: Nov. 7, 2008

Related U.S. Application Data
(63) Continuation of application No. 11/912,979.

(30) Foreign Application Priority Data
Apr. 28, 2005 (DE) 10 2005 019 753.1

(51) Int. Cl. A61B 5/02; G06F 19/00

(52) U.S. Cl. 600/490; 235/375

(57) ABSTRACT

A sphygmomanometer and a chip card for use therein. The sphygmomanometer includes a measuring device for determining blood pressure data and a control device. The control device has a chip card reader operable to read an insertable control chip card. The chip card includes data which is read by the chip card reader. A control function of the sphygmomanometer is carried out as a result of the data being read by the chip card reader.

### CROSS-REFERENCE TO PRIOR APPLICATION

This application is a continuation of U.S. application Ser. No. 11/912,979, filed on Oct. 29, 2007,: which is a U.S. National Phase application under 35 U.S.C. §371 of International Application No. PCT/EP2006/002205, filed on Mar. 10, 2006, and claims the benefit of German Patent Application No. 102005019753.1, filed on Apr. 28, 2005, which are incorporated herein. The International Application was published in German on Nov. 2, 2006 as WO 2006/114158 A1 under PCT Article 21(2).

### FIELD OF THE INVENTION

The invention relates to a sphygmomanometer with a measuring device for determining blood pressure data and a control device for controlling the measuring device and/or evaluating, processing or representing the blood pressure data. The invention also relates to a chip card for such a sphygmomanometer.

### BACKGROUND

The blood pressure data, measured by sphygmomanometers, is usually stored at the present time in volatile memories. When used by several persons, some of the data is switched over to memory banks. Along with blood pressure data, the date and time are also stored, so that the time may be allocated to the daily profile. However, with conventional sphygmomanometers, it is difficult to read this data from the sphygmomanometer and to visualize it in a clearly arranged manner in a meaningful representation. Some sphygmomanometers include an interface in order to be able to transfer data to a PC. The possibilities in this regard are, however, limited since, on the one hand, it requires an appropriate PC program in order to be able to process the blood pressure data further in the format in which it is read. On the other hand, suitable hardware is required in order to be able to read data over the interface of the sphygmomanometer.

In order to be able to store large amounts of blood pressure data, U.S. Pat. No. 6,506,162 B1 describes the use of exchangeable memory cards in the form of a chip at the instrument. With this system, larger amounts of data can be stored by exchanging memory cards. In addition, for an evaluation of the stored blood pressure data by a doctor, the memory card can be removed from the sphygmomanometer, taken to the hospital or the offices of the doctor and inserted there into a sphygmomanometer, in order to look at the stored data by means of this sphygmomanometer. However, this requires that there be a compatible sphygmomanometer at the hospital, which frequently is not the case, because consumer instruments usually are constructed differently from those present in hospitals.

Furthermore, it is frequently difficult to adapt the functionality of conventional sphygmomanometers, even if they have an exchangeable memory card. Although it would basically be possible to implement all possibly necessary functions in one sphygmomanometer, this would overload the sphygmomanometer for normal users and frequently make the handling of it unnecessarily difficult, especially for older patients. Moreover, it is obvious that, if more functions than necessary are implemented in the sphygmomanometer, the device often becomes too expensive.

### SUMMARY OF THE INVENTION

Therefore, the invention provides an improved sphygmomanometer, as well as an improved chip card, which avoid the disadvantages of the prior art. In particular, an increased variability of the application spectrum of tie sphygmomanometer is to be attained and/or a simplification of the data management is to be achieved

The present invention includes a sphygmomanometer and a chip card for use therein. The sphygmomanometer includes a measuring device for determining blood pressure data and a control device. The control device has a chip card reader operable to read an insertable control chip card. The chip card includes data which is read by the chip card reader. A control function of the sphygmomanometer is carried out as a result of the data being read by the chip card reader.

The present invention expands the functionality of the control of the sphygmomanometer by inserting a chip card. The chip card is used here not only for expanding the memory, but also for intervening in the control of the sphygmomanometer itself Pursuant to the invention, the control device has a chip card reader for reading a control chip card, which can be inserted in the sphygmomanometer. The control device is constructed so that at least one of the control functions of the control device, influencing the operation of the instrument, can be enabled, activated and/or executed only by the data read via the chip card reader. In particular, it is possible to upgrade the sphygmomanometer by inserting the appropriate chip card, that is, the extent of the performance of the device can be expanded correspondingly. In this way, it is possible to keep the basic instrument relatively simple. By buying the appropriate card functions, it can be upgraded to a higher grade instrument with additional functions.

In a further development of the invention, the appropriate control means for carrying out the additional control functions may already be implemented in the control device present in the instrument. The chip card used only reads the corresponding control data, especially a code, which activates or enables the control function present in the control device in the instrument.

Alternatively or in addition, controls may be provided on the chip card for carrying out additional control functions, so that the control device in the instrument accesses the controls stored on the chip card when implementing the respective control function, that is, the chip card becomes part of the control here.

Basically, very different control functions can be implemented by or subsequently added to the sphygmomanometer in this way. According to one embodiment of the sphygmomanometer, an alarm function may be realized at the sphygmomanometer with the help of such a control chip card. For example, an alarm device, provided at the sphygmomanometer, can be activated at times that may be programmed by the doctor, in order to remind the patient by an acoustic or vibrational alarm that the next measurement is due. Alternatively or in addition, an alarm control, which activates the alarm device when the control device detects measured values outside of a tolerance range, may be provided by using the chip card.

Alternatively or in addition, the triggering and/or evaluation of a position-determining device, for example, in the form of a tilt sensor, may also be implemented by inserting the control chip card. Such a position-determining device can establish that the sphygmomanometer is in the correct position relative to the heart, so that the control device, when the sphygmomanometer leaves the nominal position, can initiate an alarm and/or record values measured only when the nominal position has been assumed.

Alternatively or in addition, a determination of movement artifacts can also be released, activated and/or additionally provided by inserting the control chip card. Artifacts in the blood pressure data, resulting from undesirable movements or accelerations, are determined by means of a device for detecting motion and/or acceleration and can be taken into consideration when evaluating the blood pressure data.

Alternatively or additionally, it is possible to intervene in the control of the display of the sphygmomanometer by inserting a suitable control chip card. For example, an expanded display representation can be provided, which visualizes an expanded evaluation of the blood pressure data that has been ascertained. For example, additional information concerning the course of the blood pressure data over several measurements can be shown at the display, but not by the basic instrument without a chip card.

Alternatively or in addition, the memory of the sphygmomanometer can also be expanded with the chip card. In this case, the chip card reading device is constructed as a read/write device, so that the blood pressure data, determined by the measuring device, and/or data derived therefrom by the control device of the instrument, can be written in the memory of the chip card provided for this purpose.

In order to achieve better data management, especially a data evaluation in almost any office of a physician, the sphygmomanometer, according to a further aspect of the present invention, is distinguished owing to the fact that a chip card accommodating device of the sphygmomanometer is constructed in such a manner, that a chip card, standardized according to ISO/IEC 7816 (especially ISO/IEC 7816-2) and/or according to ISO7810, fits and can be inserted, and that the control device of the sphygmomanometer has a chip card read and/or write device for reading and/or writing on the standardized card with a contact arrangement of ISO-EC 7816 (especially of ISO/IEC 7816-2) and/or of ISO7810. A sphygmomanometer, constructed in such a manner, permits chip cards to be used, which are also generally for other purposes. In particular, data, written on the chip card of the sphygmomanometer, can be read by card-reading devices, which are usually present in a doctor's office for reading electronic health cards.

In a further development of the invention, a chip card read and/or write device of the sphygmomanometer is constructed in such a manner, that the blood pressure data is written in accordance with the aforementioned standards in those memory tags of the chip card, which, in accordance with the official specifications of the electronic health cards, are provided for storing patient and/or insurance data within the scope of the so-called voluntary applications. By means of this construction of the chip card write and/or read device of the sphygmomanometer, an unintended use of the readers, intended for reading the health cards in the offices of a doctor, is simplified further. In particular, it is possible to do without special software. The blood pressure data are written in the memory locations of the chip card, which are read in any event by readers for the health card. For example, the average systolic pressure can be written in the memory locations of the chip card, which is read in any case by the readers for the health card. For example, the average systolic pressure can be written in the memory location of the chip card, which, according to the official specifications of the health card, is provided for the health insurance number. Instead of the name of the insurance company, the trend of the morning values, measured between 6 a.m. and 9 a.m., can be written in the corresponding memory tag. As an explanation, abbreviations precede the corresponding numerical values and the appropriate units follow them, so that the display of the information is self explanatory and is also understandable in spite of the wrong reading template of the health card readers. The reading template of the health card reader is, as it were, misused and utilized for the rapid output of the data of the sphygmomanometers in the office of a doctor. By these means, the data inventory of the sphygmomanometer can be read in almost all doctors offices independently of the hardware present there for measuring blood pressure.

In order to ensure that the chip card of the sphygmomanometer is read by the health card reader, the chip card of the sphygmomanometer advantageously may have a code, which identifies it as a health insurance card. By these means, refusal by the health insurance card reader present in doctors' offices to read the card because the card is identified as an unknown data carrier, is prevented.

These and further distinguishing features are evident by referring to the drawing, in which:

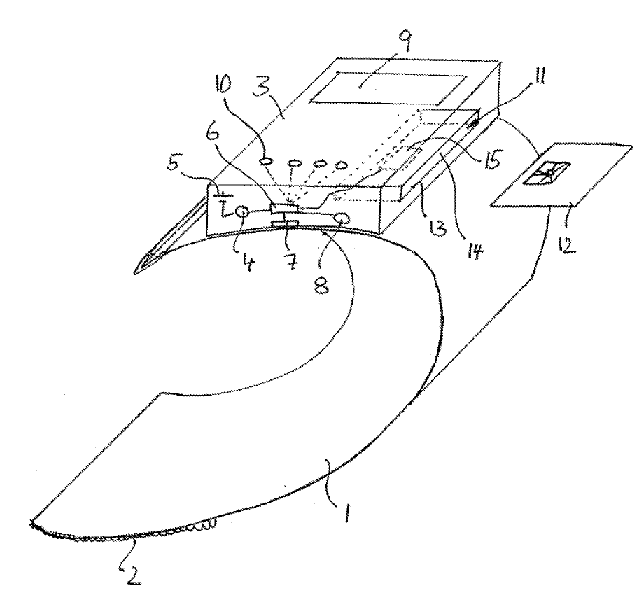

FIG. 1 shows a diagrammatic, perspective view of a wrist sphygmomanometer according to a preferred embodiment of the invention.

The sphygmomanometer, shown in FIG. 1, includes a sleeve 1, which can be placed about a wrist of a patient and fixed there, for example, by a positive hook locking mechanism 2. The sleeve 1 can be filled with a fluid and, conversely, emptied once again, in order to bring about the build up and decrease in pressure in the desired manner. For this purpose, a pump 4 is provided in the equipment housing 3, which is fastened to the upper side of the sleeve 1. The pump 4 is supplied from an energy source 5, for example, in the form of batteries or accumulators, which can be accommodated in an appropriate battery or accumulator compartment in the equipment housing 3. The pump 4 is controlled by a control device 6, which is also accommodated in the equipment housing 3, so that the sleeve 1 can be blown up and then vented once again according to a course, which can be specified by the control device.

Furthermore, the sphygmomanometer includes a pressure sensor 7, which detects pressures in the sleeve during a measuring process and emits pressure signals, which are then evaluated by the control device 6. Furthermore, a position, movement and/or acceleration sensor 8 is provided in the equipment housing 3. Using a signal from this sensor 8, a control device 6 can recognize whether the sphygmomanometer is held sufficiently close to its nominal position at the heart and sufficiently secure.

As shown in FIG. 1, a display 9 for displaying the blood pressure measured and/or for indicating signals or commands to the user, as well as several control keys 10 for inputting control commands, both of which are connected to a control device 6, are provided on the equipment housing 3.

Furthermore, the equipment housing 3 includes an interface 11, into which a chip card 12 can be inserted, with which control functions of the sphygmomanometer can be expanded and additional memory can be made available for storing blood pressure data. In the embodiment shown, an insertion slot 13 is provided in the housing 3 for the memory card 12. However, in deviation from the embodiment shown, an accommodating door or lid may also be provided, so that the chip card 12 may be inserted with its flat side. In any case, the chip card accommodating device 14 is constructed in such a manner, that a standard chip card of ISO/IEC 7816 or ISO7810 can be inserted so as to fit accurately. In the interior of the insertion slot 13, a chip card write and read device 15 is provided, which is connected with a control device 6, on the one hand, in order to transfer data from the sphygmomanometer to the chip card 12 and, conversely, to read information, stored on the chip card 12, into the sphygmomanometer.

The insertion slot for the chip card in the housing can also, alternatively, be constructed at one of the three end faces, other than the one shown here, that is, in particular, not at the side shown, at which the housing is held at the chest for the measurement.

On the one hand, the chip card 12 forms a memory device, in which the blood pressure data generated in the sphygmomanometer, can be stored. Furthermore, the chip card 12 also forms part of the control device of the sphygmomanometer. In particular, controls which can be read when the chip card is inserted and bring about enablement and/or activation and/or actualization of a control function of the control device 6, can be stored on the chip card 12. Alternatively or in addition, control algorithms, which interact with the control device 6 in the equipment housing 3 and adapt these for additional control functions, may also be stored on the chip card 12. In other words, control functions may also be processed on the chip card 12.

The chip cards 12, obtainable as refills, advantageously may have a writable field, which optionally may have a blank, so that the name of the patient and the date can be noted on the card. With the storage capacity of the chip card 12, an archive for several months of blood pressure data is available and appreciably expands the memory of the sphygmomanometer.

The data management of the sphygmomanometer can be improved considerably by the chip card 12. When the chip card is inserted, the previous data inventory can be ascertained by a program and the current memory data appended. Subsequently, the data stored in the permanent memory of the sphygmomanometer can be erased. As soon as the memory of the chip card 12 is full, an appropriate message can be displayed on the sphygmomanometer.

In order to make it easier to transfer the blood pressure data stored on the chip card 12 in a Doctor's office, the read-write device 15 of the sphygmomanometer may be constructed in such a manner, that the corresponding data is written in a memory area for patient data can be read by health card readers, which are in doctors' offices. In this way, the reading template of the office computer, which is connected to the health card reader, can be used in the manner not intended. In addition, the chip card 12 advantageously has a ROM code, which identifies the chip card in the health insurance card readers as a health insurance card.

Advantageously, the equipment properties of the sphygmomanometer can be programmed with the help of the chip card. For example, with special chip cards 12, which optionally can include RAM, control functions of the control device 6 can be re-coded so that the bothersome input over control keys 10 may be omitted. In addition, alarm functions, an expanded evaluation and a display representation of the blood pressure data, detection and consideration of position, system monitoring for artifacts during the blood pressure measurements and/or other data evaluating systems can each be added in the manner described above.

Additionally, the control device 6 can also be constructed so that, when the chip card 12 is not inserted, the sphygmomanometer is blocked and that it is enabled only when a suitable chip card 12 is inserted into the instrument. Accordingly, misuse of the instrument, for example, by children, can be prevented.

1. A sphygmomanometer comprising:
a measuring device for determining blood pressure data; and
a control device including a chip card reader operable to read an insertable control chip card,
wherein at least one control function of the control device is carried out by data read via the chip card reader.

2. The sphygmomanometer recited in claim 1, wherein a control process operable to execute the at least one control function is provided in the control device.

3. The sphygmomanometer recited in claim 1, wherein a control processoperable to execute the at least one control function is provided on the chip card and the control device is operable to access the control process stored on the chip card.

4. The sphygmomanometer recited in claim 1, wherein the at least one control function comprises triggering an alarm.

5. The sphygmomanometer recited in claim 1, wherein the at least one control function comprises evaluating a position-determining device.

6. The sphygmomanometer recited in claim 1, wherein the at least one control function comprises evaluating a movement-detection device.

7. The sphygmomanometer recited in claim 1, wherein the at least one control function comprises evaluating the determined blood pressure data and controlling a display of the sphygmomanometer.

8. The sphygmomanometer recited in claim 1, wherein the chip card reader comprises a read/write device operable to write data from the control device onto the chip card (12).

9. The sphygmomanometer recited in claim 1, wherein the control device is operable to store blood pressure data on the chip card.

10. The sphygmomanometer recited in claim 1 further comprising a chip card accommodating device operable to hold a chip card that is standardized in accordance with ISO/IEC 7816 and/or ISO7810.

11. The sphygmomanometer recited in claim 8, wherein the read/write device is operable to write blood pressure data in memory tags of an ISO/IEC 7816 and/or ISO7810 chip card.

12. A chip card for a sphygmomanometer, comprising control data operable to carry out a control function of the sphygmomanometer when the control data is read by a card reader of the sphygmomanometer.

13. The chip card recited in claim 12, wherein the chip card is constructed in accordance with at least one of ISO/IEC 7816 and ISO7810.

14. The chip card recited in claim 12, further comprising a control process operable to trigger an alarm of the sphygmomanometer.

15. The chip card recited in claim 12, further comprising a control process operable to evaluate at least one of a position detector and a movement detector.

16. The chip card recited in claim 12, further comprising a control process operable to evaluate blood pressure data and to control a display of the sphygmomanometer for displaying information related to a blood pressure evaluation.

17. The chip card recited in claim 12, further comprising a memory operable to store blood pressure data.

18. The sphygmomanometer recited in claim 1, further comprising the chip card.
